(19) United States
(12) Patent Application Publication
(10) Pub. No.: US 2025/0325733 A1
(43) Pub. Date: Oct. 23, 2025

(54) Multifunctional Surface Modification of Biomaterials to Reduce Thrombosis

(71) Applicant: McMaster University, Hamilton (CA)

(72) Inventors: Anthony Chan, Ancaster (CA); John Brash, Ancaster (CA); Kyla Sask, Waterdown (CA); Ravi Selvaganapathy, Dundas (CA); Christoph Fusch, Feucht (DE); Gerhard Fusch, Ancaster (CA); Niels Rochow, Nurnberg (DE); Salhab El Helou, Hamilton (CA); Siyuan Li, Hamilton (CA)

(21) Appl. No.: 18/868,406

(22) PCT Filed: May 24, 2023
(86) PCT No.: PCT/CA2023/050710

§ 371 Date: Nov. 22, 2024

Related U.S. Application Data
(60) Provisional application No. 63/345,221, filed on May 24, 2022.

(51) Int. Cl. A61L 33/00

(52) U.S. Cl. CPC A61L 33/0011; A61L 33/0082

(57) ABSTRACT

An antithrombotic surface-modified biomaterial is provided comprising a biomaterial substrate coated with a polymerizable dopamine-containing bioadhesive to which is attached one or more antithrombotic agents. A method of preparing the surface-modified biomaterial is also provided. The surface-modified biomaterial is beneficial for use in blood-contacting medical devices such as catheters, dialyzers and blood oxygenators to prevent or minimize the occurrence of thrombosis on the surface thereof.

### FIELD

The present application relates to biomaterial surface modification and, in particular, to modification with one or a combination of bioactive agents to reduce thrombosis.

### BACKGROUND

Blood contacting medical devices continue to require systemic anticoagulant and antiplatelet therapies with their use due to the initiation of coagulation and thrombosis at the surface of the biomaterial. This occurs due to rapid protein adsorption to the material surface, followed by platelet adhesion and other cellular interactions. Various synthetic polymers have commonly been used for blood-contacting devices, including polyurethanes, polydimethylsiloxane (PDMS), polymethylmethacrylate (PMMA), polytetrafluoroethylene (ePTFE), poly (ethylene terephthalate) (PET) and others. Without modifying the surface of these materials, uncontrolled protein adsorption will occur, leading to complications and failure of devices such as catheters, blood oxygenators, hemodialyzers and vascular grafts.

Many single surface modification strategies have been applied to polymers to reduce thrombus formation, including immobilization of anticoagulants such as heparin and hirudin. However, these technologies still suffer from various limitations. Fibrinolytic surface modifications that provide the ability to lyse fibrin clots can also be applied to materials by incorporating molecules such as tissue plasminogen activator (t-PA). An antithrombin-heparin complex (ATH) is a novel anticoagulant with a longer half-life and higher inhibition rate than heparin. The AT portion of ATH is covalently linked to heparin and is preactivated to allow rapid inhibition for thrombin or factor Xa directly. The heparin portion of ATH can also bind to AT in plasma. Thus, it can inhibit coagulation by both direct and catalytic heparin-based mechanisms.

Polydopamine (PDA) is a recently discovered “bio-glue” with strong adhesive characteristics similar to mussel adhesive properties. Oxidation occurs when dopamine is exposed to alkaline conditions in an aqueous solution. Following this, self-assembled PDA layers form on organic or inorganic surfaces in contact with the solution. PDA has been widely used for surface functionalization of biomolecules for easy fabrication and biocompatibility.

It would be desirable to develop improved surface modified biomaterials that reduce thrombosis.

### SUMMARY

A new approach to modifying biomaterial surfaces with two or more antithrombotic bioactive molecules has been developed to provide a biomaterial with anticoagulant, fibrinolytic and/or antiplatelet functions which achieves enhanced efficacy in reducing thrombosis.

Thus, in one aspect, an antithrombotic surface-modified biomaterial is provided comprising a biomaterial substrate coated with a polymerizable dopamine-containing coating to which is attached one or more antithrombotic agents.

In another aspect, a method of preparing an antithrombotic surface-modified biomaterial is provided comprising the steps of i) contacting a biomaterial substrate with a dopamine-containing bioadhesive under conditions sufficient to polymerize the bioadhesive as a coating on the biomaterial substrate, and ii) contacting the polymerized dopamine-containing coated biomaterial with a quantity of one or more antithrombotic agents.

Embodiments of the invention are described in the detailed description that follows by reference to the following figures.

### BRIEF DESCRIPTION OF THE FIGURES

FIG. 1 shows a schematic illustration of the modification process to form the dual-functional coatings. A) PDMS-PDA-ATH-t-PA, B) PDMS-PDA-ATH/t-PA and C) PDMS-PDA-t-PA-ATH.

FIG. 2 shows the water contact angle measurements of PDMS, and various PDMS-PDA modified surfaces.

FIG. 3 shows the surface density of ATH (a) and t-PA (b) immobilized on the material surface along with values after desorption into plasma and further desorption after 4 days. ATH and t-PA were radiolabeled with I¹²⁵ separately and measured with a gamma counter.

FIG. 4 shows a) the AT adsorption from plasma and b) the Anti-FXa activity on control and modified discs.

FIG. 5 shows the t-PA activity on modified surfaces a) before plasma and b) after overnight plasma incubation.

FIG. 6 shows the coagulation time on a) PDMS, PDMS-PDA and control discs, b) PDMS-PDA-ATH and PDMS-PDA-t-PA discs, and C) PDMS-PDA-t-PA-ATH, PDMS-PDA-ATH-t-PA and PDMS-PDA-ATH/t-PA modified discs of recalcified citrated plasma. Clot formation is seen as an increase in absorbance and clot lysis as a decrease in absorbance.

FIG. 7 shows devices for oxygenator modification (a), including a peristaltic pump, oxygenator unit and connected tubing, and (b) oxygenator before and after PDA modification for 24 hours.

FIG. 8 shows the coagulation time of plasma in the modified oxygenator unit. Clot formation is seen as an increase and clot lysis as a decrease in pressure

### DETAILED DESCRIPTION

A novel antithrombotic surface-modified biomaterial is provided comprising a biomaterial substrate coated with a polymerized dopamine-containing bioadhesive to which is attached antithrombotic agents.

The biomaterial substrate to be surface-modified in accordance with embodiments of the present invention may be any substance or material that is suitable for use in medical or biological applications. Thus, the biomaterial substrates are appropriate to interact with biological systems, such as tissues, cells and/or bodily fluids, with the aim of providing therapeutic or diagnostic outcomes. The biomaterial substrate can take various forms, including solids or gels, and they may be natural or synthetic in origin. Examples of biomaterial substrates suitable for use include, but are not limited to, synthetic polymers such as polyurethanes, polydimethylsiloxane (PDMS), polymethylmethacrylate (PMMA), polytetrafluoroethylene (ePTFE), poly (ethylene terephthalate) (PET), polysulfone, polyethersulfone (PES), polypropylene, polyethylene, polyvinylidene fluoride (PVDF), polyvinylchloride (PVC), polyamide and polyetheretherketone (PEEK). Metal substrates include, but are not limited to, titanium, titanium alloys, stainless steel, cobalt chromium, cobalt alloys and nitinol. Natural biomaterial substrates include, but are not limited to, biopolyesters such as polylactic acid (PLA), polyhydroxyalkanoates (PHAs) and their derivatives, polysaccharides such as hyaluronic acid (HA), alginate, cellulose and chitosan, as well as polypeptides and proteins such as collagen, gelatin, fibrin, poly-glutamic acids (PGA) and antimicrobial peptides (AMPs).

The selected biomaterial substrate is coated with a polymerizable dopamine-containing bioadhesive. The term “bioadhesive” refers to a substance that can adhere to the substrate and biomolecules such as antithrombotic agents, is itself biocompatible and exhibits wettability or a capacity to disperse to create a binding surface. Examples of dopamine-containing bioadhesives include but are not limited to polydopamine (PDA), mussel foot proteins (mfps), including mfp-1, mfp-2, mfp-3, mfp-4, mfp-5 and mfp-6, and derivatives thereof.

A coating of a dopamine-containing bioadhesive, such as a PDA coating, is formed on the surface of the biomaterial substrate as a “bio-glue” to attach the biomolecules to the substrate by immersing the biomaterial substrate in a solution of the bioadhesive under conditions suitable for the bioadhesive to polymerize and form a coating on the substrate. As one of skill in the art will appreciate, the coating conditions may vary with the selected dopamine-containing bioadhesive as well as the selected biomaterial substrate. In embodiments, incubation of the substrate with the bioadhesive for a period of time at room temperature, e.g. for about 24 hours, is sufficient to achieve a suitable bioadhesive coating on the substrate. Preferably, an amount of bioadhesive is used which substantially coats the biomaterial substrate. In an embodiment, an amount of bioadhesive is used which fully coats the biomaterial substrate.

Antithrombotic agents are then attached to the bioadhesive coating on the biomaterial substrate. Attachment of antithrombotic agents to the bioadhesive may be by any one or more of the following: covalent bond, ionic bond, hydrogen bond, Van der Waals forces, electrostatic attraction and metallic bonds. Examples of antithrombotic agents include anticoagulant agents, fibrinolytic agents and antiplatelet agents. The term “anticoagulant agent” refers to an agent that inhibits the clotting of blood by interfering with the blood coagulation process. Anticoagulants are used to prevent or treat conditions characterized by excessive blood clot formation or to minimize the growth of existing blood clots. The blood clotting process involves a series of complex interactions among different coagulation factors in the blood and anticoagulant agents can target specific steps in this process including: enhancing natural anticoagulants, inhibiting clotting factor activity and inhibiting clotting factor synthesis.

Anticoagulants that act by enhancing the activity of natural anticoagulant proteins in the blood, such as antithrombin, interact with antithrombin and enhance its ability to inhibit clotting factors, including thrombin and factor Xa. Examples of anticoagulant agents, include heparin, low molecular weight heparin (LMWH) and fondaparinux, as well as antithrombin itself, and complexes formed with antithrombin such as antithrombin-heparin complexes.

Anticoagulants that inhibit clotting factor activity, for example direct oral anticoagulants (DOACs), target specific clotting factors directly to inhibit their activity. Examples of DOACs include rivaroxaban, apixaban, betrixaban and edoxaban, which inhibit factor Xa (FXa), and dabigatran which inhibits factor IIa (thrombin). Other anticoagulant agents that act by inhibiting clotting factor activity include hirudin, bivalirudin, argatroban, thrombomodulin and corn-trypsin inhibitor (CTI).

Anticoagulants which act by inhibiting the synthesis of certain clotting factors in the liver, for example, vitamin K-dependent clotting factors (II, VII, IX, and X). Examples of such anticoagulants include vitamin K antagonists (VKAs) such as warfarin. By reducing the level of clotting factors, the ability of the blood to clot is diminished.

The term “fibrinolytic agents” refers to an agent that promotes the breakdown of blood clots by activating the fibrinolytic system. The fibrinolytic system is a natural process that functions to dissolve blood clots that have formed within blood vessels. Fibrinolytic agents work by activating plasminogen, which is present in the blood and converting it into the active enzyme plasmin, which breaks down fibrin, the main component of blood clots. Examples of fibrinolytic agents include tissue plasminogen activator (t-PA) and urokinase plasminogen activator (u-PA).

The term “antiplatelet agents” refers to an agent that inhibits the activation and aggregation of platelets in the blood. Platelets are small cell fragments involved in blood clotting, and their aggregation plays a crucial role in the formation of blood clots. Antiplatelet agents work by interfering with various stages of platelet activation and aggregation, ultimately reducing the risk of unwanted blood clot formation. They are commonly prescribed to prevent or treat conditions such as cardiovascular diseases, including heart attacks and strokes, where excessive clotting can be detrimental to health. Examples of antiplatelet agents include aspirin, adenosine diphosphate (ADP) receptor inhibitors, such as clopidogrel, ticagrelor and prasgrel, and GPIIb/IIIa inhibitors, such as tirofiban, abciximab and eptifibatide. Other antiplatelet agents include dipyridamole, prostacyclin and apyrase.

An antithrombotic agent, such as an anticoagulant agent, an antifibrinolytic agent and/or an antiplatelet agent is immobilized on the bioadhesive coating of the biomaterial substrate by incubating the substrate with the antithrombotic agent under conditions suitable to immobilize the antithrombotic agent on the coating. In one embodiment, a single type of antithrombotic agent is immobilized on the coated biomaterial substrate. In other embodiments, two or more different antithrombotic agents are immobilized on the coated biomaterial substrate, which may be of the same type, i.e. each are anticoagulant agents, each are antifibrinolytic agents, or each are anti-platelet agents. Alternatively, the two or more antithrombotic agents comprise different types, i.e. an anticoagulant agent and a fibrinolytic agent or an antiplatelet agent; a fibrinolytic agent and an antiplatelet agent; or comprise each of an anticoagulant agent, a fibrinolytic agent and an antiplatelet agent.

Thus, in one embodiment, an anticoagulant, such as heparin or ATH, is immobilized on the coated biomaterial substrate, e.g. a PDA-coated substrate, by incubating the PDA-modified biomaterial substrate in a heparin or ATH solution. The coated biomaterial substrate modified with anticoagulant is then incubated in a fibrinolytic molecule solution such as t-PA to form a dual-functional antithrombotic coating. The amount of each antithrombotic agent utilized to modify the surface of the coated substrate is an amount sufficient to yield a functional surface-modified biomaterial, i.e. an amount that yields surface-modified biomaterial that exhibits antithrombotic activity. The amount of antithrombotic agent(s) will vary with the antithrombotic agent or agents utilized. In the case of two antithrombotic agents, the amounts of each used may vary. In embodiments of the invention, the amounts of each may be about equal or may be in a ratio of 75:25 to 25:75, or 60:40 to 40:60. In the case of more than two antithrombotic agents, equal amounts of each may be used, or greater amounts of one or more of the antithrombotic agents.

Efficacy of the surface-modified biomaterial in accordance with an aspect of the present invention is determined by determining the antithrombotic activity of the biomaterial, i.e. the ability of the modified biomaterial to prevent or minimize thrombosis. For example, the surface-modified biomaterial may be incubated with plasma at 37° C. to determine if any clotting of the plasma at the surface of the biomaterial occurs. No clotting indicates that the surface-modified biomaterial possesses antithrombotic activity. If clotting does occur, the surface-modified biomaterial may still possess antithrombotic activity as evidenced by clot lysis within a period of time, e.g. within 6-8 hours, preferably within less than 2-3 hours.

The method of preparing an antithrombotic surface-modified biomaterial in accordance with another aspect of the invention comprises the steps of: i) contacting a biomaterial substrate with a dopamine-containing bioadhesive under conditions sufficient to polymerize the bioadhesive as a coating on the biomaterial substrate, and ii) contacting the polymerized dopamine-containing coating with a quantity of one or more antithrombotic agents under conditions sufficient to achieve attachment of the antithrombotic agent.

The order that the bioadhesive-coated biomaterial substrate is modified with two or more antithrombotic agents may be varied. By adjusting the modification order of the biomaterial and quantity of the antithrombotic agent(s), the surface density and stability of the immobilized thrombotic agents can be controlled on the surface of the substrate. For example, the coated biomaterial substrate may be contacted with two or more selected antithrombotic agents simultaneously. Alternatively, coated biomaterial substrate is contacted with two or more selected antithrombotic agents sequentially, i.e. the substrate is contacted with one antithrombotic agent at a time. In one embodiment, the antithrombotic agents comprise an anticoagulant agent and a fibrinolytic and/or an antiplatelet agent, and the coated biomaterial substrate is contacted with the anticoagulant agent first, followed by sequential contact of the coated biomaterial substrate with the fibrinolytic and/or antiplatelet agents.

In an exemplary embodiment, the surface coating formed by modifying with an anticoagulant agent, such as ATH, first does not form a monomolecular layer on the entire surface of the substrate. Therefore, there is bioadhesive coating surface available for modification (attachment) by one or more other antithrombotic agents such as a fibrinolytic agent, such as t-PA, and/or an anti-platelet agent, directly on the surface of the substrate. The formation of the anticoagulant coating (e.g. ATH) also changes the surface morphology, resulting in significant physical adsorption of the fibrinolytic agent (e.g. t-PA) on the surface. As a result, a higher density, stable fibrinolytic coating (e.g. t-PA) on the surface is achieved, which is greater than modification with fibrinolytic agent alone. Although physically adsorbed antithrombotic agents such as t-PA may detach in a complex blood environment, a sufficient amount of t-PA adsorbs onto the coated surface to form a stable, functional coating. When the coated substrate is initially modified with a fibrinolytic agent such as t-PA (e.g. at a density of 0.1 mg/mL), it forms a monomolecular layer on the surface, and subsequent modifications with different antithrombotic biomolecules such as anticoagulant agents (e.g. ATH) or antiplatelet agents result in subsequent attachment to the monolayer surface (i.e. the surface of the fibrinolytic agent monolayer) and expression of their respective functions on the surface of the fibrinolytic (e.g. t-PA) layer.

The order of antithrombotic agent modification to the coated substrate and quantity of each antithrombotic agent can also affect the activity of each antithrombotic agent. For example, ATH modification after t-PA modification achieves a lower grafting density on the surface but exhibits higher activity because such modification leads to changes in the protein conformation or orientation of ATH, which, upon contact with t-PA, undergoes alterations to expose more active sites. The surface morphology changes caused by the modification may also enhance the physical adsorption of the thrombotic agent(s).

The present surface-modified biomaterial is useful for the modification of medical devices. The term “medical device” refers to a health or medical instrument, apparatus or implant used in the diagnosis, treatment or prevention of a disease or physical condition. Of particular interest are blood-contacting medical devices including, but are not limited to, catheters, guidewires, dialyzers, oxygenators (artificial lungs), heart-supporting systems, cardiac pacemakers, vascular grafts, stents, heart valves, blood pumps, sutures, micro-, and nanoparticles, scaffold for containing cells or tissue, orthopedic implants and dental implants.

For example, a microfluidic oxygenator made with a biomaterial substrate such as PDMS can be modified according to the present invention to achieve a device having antithrombotic effects. The term “oxygenator,” as used herein, refers to a heart-lung machine connected to the heart by drainage tubes that divert blood from the venous system, directing it to an oxygenator. The oxygenator removes carbon dioxide and adds oxygen to the blood, which is then returned to the body's arterial system. For devices like microfluidic oxygenators, a bioadhesive coating is applied to the blood-contacting surface of the device by circulating a solution of the bioadhesive within the fluidic system, followed by circulating one or more selected antithrombotic agents, e.g. anticoagulant, fibrinolytic, and/or antiplatelet agent solutions, within the system to achieve antithrombotic surface modification.

Unless otherwise indicated, the definitions and embodiments described in this and other sections are intended to apply to all embodiments and aspects of the present application herein described. They are suitable as would be understood by a person skilled in the art.

In understanding the scope of the present application, the term “comprising” and its derivatives, as used herein, are intended to be open-ended terms that specify the presence of the stated features, elements, components, groups, integers, and steps, but do not exclude the presence of other unstated parts, elements, components, groups, integers and/or steps. The foregoing also applies to words with similar meanings, such as “including,” “having,” and their derivatives. The term “consisting” and its derivatives, as used herein, is intended to be closed terms that specify the presence of the stated features, elements, components, groups, integers, and steps but exclude the presence of other unstated features, elements, components, groups, integers and/or steps. The term “consisting essentially of,” as used herein, is intended to specify the presence of the stated features, elements, components, groups, integers, and/or steps, as well as those that do not materially affect the essential and novel characteristic(s) of features, elements, components, groups, integers, and steps.

Terms of degree such as “substantially,” “about,” and “approximately” as used herein mean a reasonable amount of deviation of the modified time such that the result is not significantly changed. These terms of degree should be construed as including a deviation of at least ±10% of the modified term if this deviation would not negate the meaning of the word it modifies.

As used in this application, the singular forms “a,” “an,” and “the” include plural references unless the content clearly dictates otherwise.

In embodiments comprising an “additional” or “second” component, the second component as used herein is chemically different from the other components or first component. A “third” component is different from the other first and second components, and further enumerated, or “additional” components are similarly different.

The term “and/or” as used herein means that the listed items are present, or used, individually or in combination. This term means that “at least one of” or “one or more” of the listed items is used or present.

Embodiments of the invention are described by reference to the following specific examples which are not to be construed as limiting.

### EXAMPLES

### Example 1

### Preparation of Polydimethylsiloxane (PDMS) Discs

Materials: Polydimethylsiloxane (PDMS) discs were prepared using the Sylgard® 184 Silicone Elastomer kit from Dow Corning (Midland, MI) with a 10:1 ratio of elastomer base to curing agent. The components were poured into a Petri dish to form a thin layer, placed under a vacuum for 30 min to remove air bubbles, then cured at 60° C. in an oven for 8 hours. PDMS, approximately 0.5 mm thick, was cut into discs using a 6 mm diameter punch.

### Example 2

### Surface Modification of Polymers

Dopamine hydrochloride was obtained from Sigma-Aldrich (Oakville, ON). ATH was prepared and obtained from Dr. Anthony Chan's group at the Thrombosis & Atherosclerosis Research Institute, as described in previously (Patel et al. Thromb Res 2007, 120 (2): 151-160). Briefly, anti-thrombin (AT) and heparin were incubated for 14 days at 40° C., at a molar ratio of 1:200. Next, NaBH₃CN was added and incubated for 5 h at 37° C. In a two-step procedure, ATH was purified by butyl agarose and DEAE Sepharose chromatography. T-PA (alteplase) was obtained from the Hamilton Health Sciences Pharmacy Services at the McMaster University Medical Centre (Hamilton, ON).

To prepare the polydopamine (PDA) coated samples, 1 mg/mL dopamine hydrochloride solution was prepared in Tris-HCl buffer at pH 8.5. PDMS discs were placed in a 96-well plate. 200 μL dopamine hydrochloride solution was added to each well. PDMS discs were incubated for 24 hours at room temperature, allowing the PDA coating to form on the PDMS surface. After polymerization, the discs were rinsed three times (5 min each time) in phosphate-buffered saline (PBS), pH 7.4. For the PDMS-PDA-ATH samples, 0.1 mg/mL ATH solution was prepared in phosphate-buffered saline (PBS, pH 7.4). PDMS-PDA discs were incubated in 0.1 mg/mL ATH solution for 24 hours at room temperature. After polymerization, the discs were rinsed three times (5 min each time) in PBS, pH 7.4.

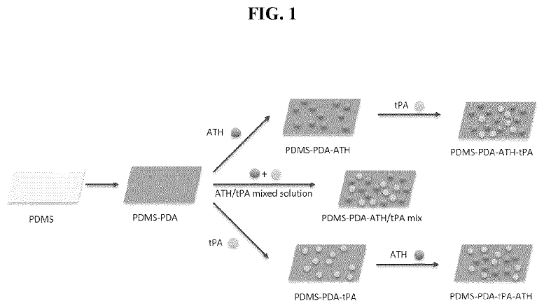

The various PDMS-PDA-ATH+t-PA dual modified samples were prepared with 0.1 mg/mL of ATH and t-PA as mixed solutions in phosphate-buffered saline (PBS, pH 7.4). As shown in FIG. 1, the dual modified samples were prepared according to three different processes:

1) PDMS-PDA-ATH-t-PA: PDMS-PDA discs were incubated in 0.1 mg/mL ATH solution for 24 hours, followed by 0.1 mg/mL t-PA solution for 24 hours at room temperature. After polymerization, the discs were rinsed three times (5 min each time) in PBS, pH 7.4 (FIG. 1A);

2) PDMS-PDA-t-PA-ATH: PDMS-PDA discs were incubated in 0.1 mg/mL t-PA solution for 24 hours followed by 0.1 mg/mL ATH solution for 24 hours at room temperature. After polymerization, the discs were rinsed three times (5 min each time) in PBS, pH 7.4 (FIG. 1C); and

3) PDMS-PDA-ATH/t-PA Mix: PDMS-PDA discs were incubated in 0.1 mg/mL of ATH and 0.1 mg/mL t-PA mixed solution for 24 hours in room temperature. After polymerization, the discs were rinsed three times (5 min each time) in PBS, pH 7.4 (FIG. 1B).

### Example 3

### Surface Characterization

Water contact angle measurements were performed to determine changes in the surface wettability of control PDMS and surface-modified PDMS.

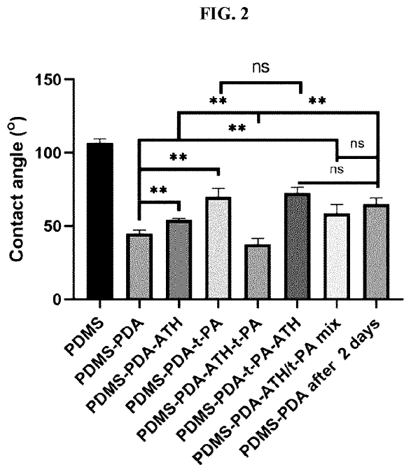

Results are shown in FIG. 2. The contact angle of unmodified PDMS is above 100° as expected for this hydrophobic material. After modification with the PDA coating, the contact angle decreased to 45°, demonstrating an increase in hydrophilicity and confirming the modification with PDA. After ATH modification, there was a small increase in contact angle to 54° and after t-PA modification there was an increase to 65°. The contact angle of PDMS-PDA-ATH-t-PA was 37°, however, modification with t-PA first followed by ATH showed no significant difference compared to PDMS-PDA-t-PA samples. The contact angle of samples modified with the mixed solution were also not significantly different from the other samples. The contact angles of PDA modified samples increased to 60° after two days, similar to dual-modified samples. Since it usually takes two days to achieve a dual-modified coating, the increase in contact angle may be a result of the modification or the change in the PDA over time. Overall, the results provide confirmation of the surface modifications.

### Example 4

### Surface Density of ATH and tPA

¹²⁵I radiolabeling was used to determine the density of ATH and t-PA on the various modified surfaces, and their surface stability was estimated via resistance to sodium dodecyl sulphate (SDS) elution.

The proteins were labelled with Na¹²⁵I using the Iodogen method. 1-2 mg of protein and five μL Na¹²⁵I solution were mixed in an Iodogen vial coated with 25 μg iodination reagent and reacted for 15 mins. The mixed reaction solution was dialyzed for 48 hours to remove the unbound isotope. Labelled protein (10%) was mixed with unlabelled protein as a tracer to determine the quantity of protein modified on the surface. After modification, the discs are washed with PBS three times to remove the unbound protein. The radioactivity was determined using a Wizard Automatic Gamma Counter from Perkin Elmer (Waltham, MA).

Surface density of ATH and t-PA on the modified discs was determined after initial uptake, and desorption of ATH and t-PA from the discs was measured following incubation in 100% plasma overnight at room temperature with shaking at 200 RPM, and following further incubation in 100% plasma after 4 days at room temperature with shaking at 200 RPM.

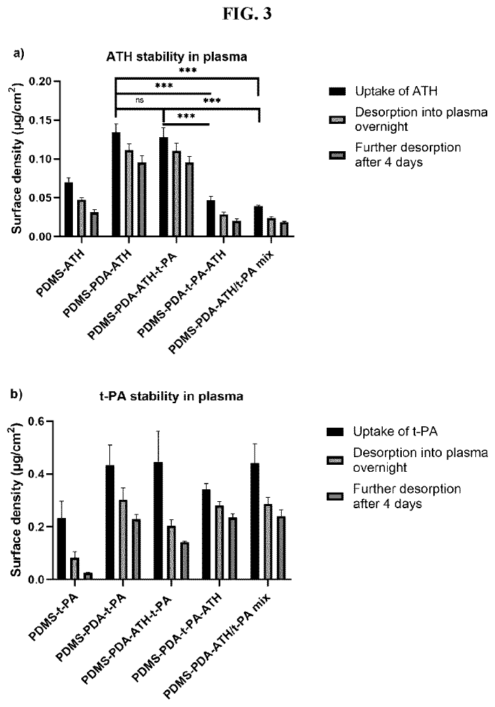

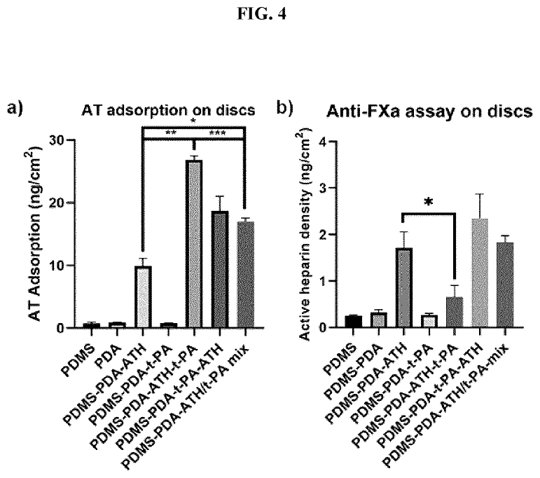

Results are shown in FIG. 3. The surface density of ATH on the PDMS surface showed significant differences with the three types of modification processes (i.e. processes (1), 2) and 3) as set out in Example 2).

For ATH, samples modified with t-PA first, then ATH, and samples modified with the mixed solution of t-PA and ATH have a very low density of ATH. Samples modified with ATH first and then t-PA showed similar ATH surface density and stability as compared to the ATH-modified sample upon initial uptake and following plasma incubation (see FIG. 3a).

For t-PA, as shown in FIG. 3b, samples modified with ATH first and then t-PA (PDMS-PDA-ATH-t-PA) showed the highest initial t-PA surface density, even higher than the sample modified with t-PA only. However, t-PA surface density significantly decreased to 0.18 μg/cm² after plasma overnight incubation (55% lost) suggesting only physical adsorption of t-PA on the surface as opposed to covalent linkage. As a comparison, samples modified with t-PA then ATH had a stable t-PA coating after plasma incubation because the t-PA was attached on the PDA coating directly. The t-PA surface density on samples modified with the ATH/t-PA mixed sample exhibited similar t-PA surface density to the PDMS-PDA-ATH-t-PA sample; however, after overnight incubation in plasma, ˜45% of t-PA was lost from the surface of the ATH-t-PA sample.

### Example 5

### ATH Activity on Discs

AT uptake from plasma and an anti-FXa assay were performed to assess the activity of ATH on discs.

Radiolabeled AT was added to plasma at 10% of normal physiological level. All modified samples were exposed to AT-radiolabeled plasma for 3 h and AT adsorption was determined.

As shown in FIG. 4a, the AT concentration on PDMS, PDMS-PDA and PDMS-PDA-t-PA samples was very low since there was no ATH modification on the surface. The AT adsorption surface density on PDMS-PDA-ATH is 10 ng/cm², indicating a specific interaction between the heparin component of ATH and AT, and was significantly lower than other dual modified samples. However, no significantly higher active heparin was observed on the dual-modified samples (as shown in the anti-FXa assay) which suggests physical adsorption of AT on the dual modified sample surface.

When an AT-heparin complex is formed it can react with FXa and the remaining FXa can be measured by the chromogenic substrate, CBS 31.39. An anti-FXa assay was used to determine the active heparin density on the discs prepared according to Example 2.

As shown in FIG. 4b, the control surface of a disc without ATH modification, had a very low active heparin density, i.e. PDMS, PDA and PDMS-PDA-t-PA. The active heparin density of an ATH modified disc, i.e. PDMS-PDA-ATH, was 1.6 ng/cm². The active heparin density of PDMS-PDA-ATH-t-PA was significantly lower (about 0.5 ng/cm²) than the PDMS-PDA-ATH disc. This suggests that t-PA modification following ATH modification decreases the ability of ATH to interact with heparin. The ATH surface density on the PDMS-PDA-ATH surface is significantly higher than PDMS-PDA-t-PA-ATH as discussed above, however the PDMS-PDA-t-PA-ATH has a higher active heparin density than PDMS-PDA-ATH. This may be because the ATH has greater exposure for active heparin or excess ATH on the surface relative to the AT used in this assay. It is expected that this effect may also take place with other anticoagulant molecules, followed by other fibrinolytic molecules and antiplatelet agents. No significant difference was observed on the PDMS-PDA-ATH/t-PA mixed surface compared to PDMS-PDA-ATH.

### Example 6

### t-PA Activity on PDMS Discs

The chromogenic substrate S2288 is cleaved by active t-PA, and thus its activity is determined by measuring the color change due to the reaction between t-Pa and S2288.

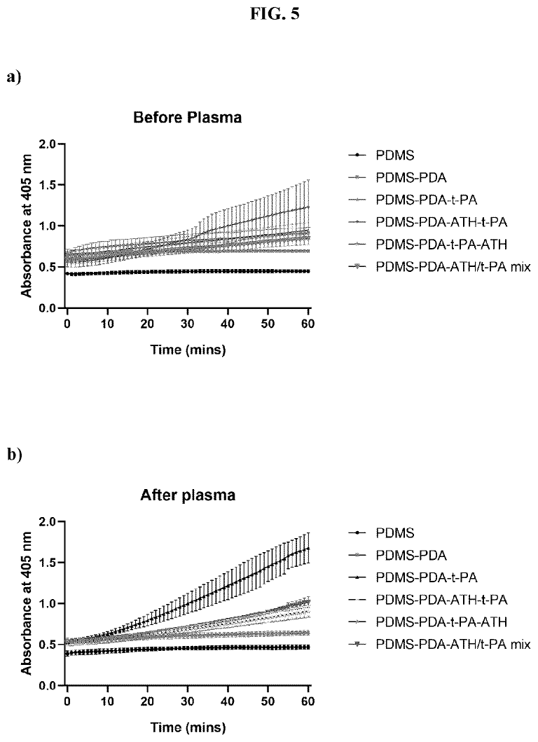

Chromogenic substrate S2288 solution (200 μL, five mM in PBS, pH 7.4) was placed in 96-well plate wells. Modified discs were incubated in the wells, and the absorbance at 405 nm was measured at 1 min intervals for 60 mins. As shown in FIG. 5a, the samples modified with ATH first, followed by t-PA, had the highest activity, unexpectedly even higher than the t-PA modified sample. The other modifications of dual-functional coatings decreased the t-PA activity on the surface, specifically, the t-PA-ATH samples. After overnight plasma incubation, all dual-modified samples showed a similar absorbance curve, indicating a loss of t-PA on the PDMS-PDA-ATH-t-PA surface.

However, t-PA activity on the PDMS-PDA-t-PA surface increased after plasma incubation (see FIG. 5b). A possible reason for this is a conformational change of t-PA from single-chain t-PA to double-chain t-PA that has a faster reaction rate with S-2288. This suggests that the t-PA activity could have increased due to the physical adsorption of t-PA after ATH modification.

### Example 7

### Clotting Time Test of Modified PDMS Discs in Plasma

Before tests, citrated plasma and 25 mM calcium chloride were kept in a 37° C. water bath. Next, disc samples were placed in 96-well plate wells, and citrated plasma was recalcified with 25 mM calcium chloride at a ratio of 1:1 (v/v, total 300 μL per well). Three wells with recalcified plasma were left as controls. The 96-well plate was shaken for 60 s, and the absorbance at 405 nm was recorded for 90 min at 1 min intervals at 37° C. in a plate reader. An increase in absorbance is indicative of clotting, while a decrease is indicative of lysis.

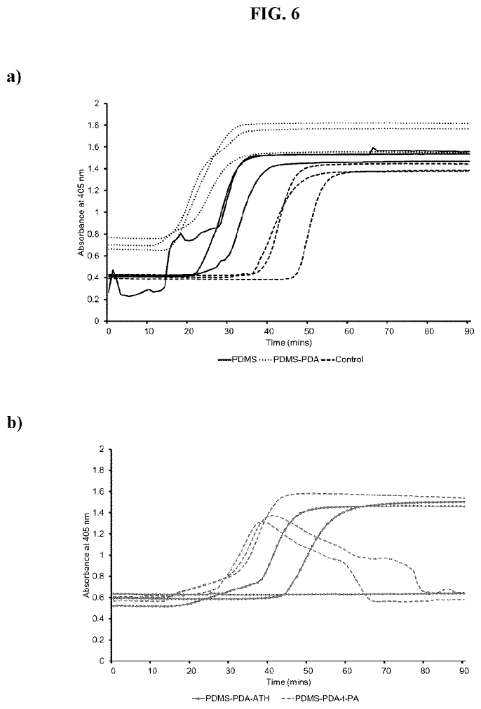

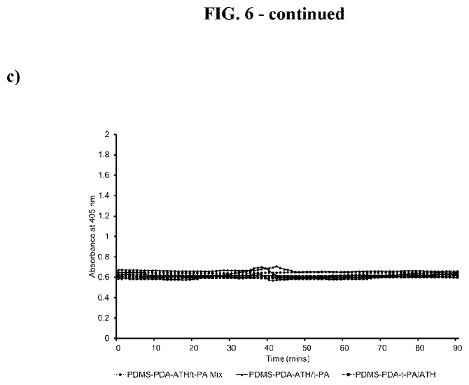

Results are shown in FIG. 6.

Unmodified PDMS, PDMS-PDA and the control formed clots but did not lyse clots within 90 min (FIG. 6a). It's important to note that the time for PDMS-PDA to start forming a clot is ˜15 min. In comparison, the unmodified PDMS and control are ˜20 min and ˜35 min, respectively, demonstrating that protein adsorption to PDA surfaces is likely to accelerate the clot formation process.

For PDMS discs modified with t-PA using PDA as a binding agent, two samples started to form clots after ˜15 min and continued until ˜40 min. After that, the absorbance decreased gradually until ˜75 min, indicating clot lysis. These data suggest that t-PA on the surface may convert plasminogen close to the surface or adsorbed on the surface to plasmin and lyse clots. One of the PDMS-PDA-ATH samples didn't form a clot in 90 min, suggesting that ATH may be able to inhibit thrombus formation on the surface. However, one of the t-PA samples and two ATH samples formed clots after ˜40 min and did not lyse clots. This indicates that the samples with ATH or t-PA modification may be variable and unstable (see FIG. 6b).

PDMS discs were dual-modified with ATH and t-PA simultaneously using mixed solutions or sequentially with the ATH solution followed by the t-PA solution and vice versa as set out in Example 2. Molecular mechanisms of ATH and t-PA binding with the PDA coating may cause a different ratio and activity of ATH and t-PA on the surface and, thus, another anticoagulant function. All dual-modified samples didn't form clots in 90 min (see FIG. 6c), demonstrating a more stable antithrombotic function than ATH or t-PA samples alone.

### Example 8

### Modification of Oxygenators

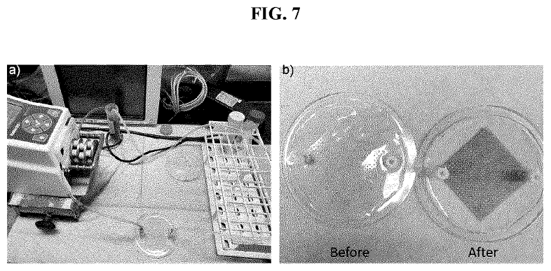

PDMS-PDA oxygenators: Surface modification of oxygenators were carried out under flow conditions as shown in FIG. 7a. 50 mL of a 1 mg/mL dopamine hydrochloride solution in Tris-HCl buffer, pH 8.5, was prepared in a 50 mL centrifuge tube. The solution was circulated through the oxygenator using a peristaltic pump at 3 mL/min for 24 h. The solution was returned to the tube, creating a closed loop. After PDA polymerization, the oxygenator was washed three times for 5 min each by circulating with PBS, pH 7.4 and followed by using a 3 mL syringe to remove loosely bound molecules. A PDA polymerized oxygenator is shown in FIG. 7b.

PDMS-PDA-ATH oxygenator: 50 mL of a 0.1 mg/mL ATH solution in PBS, pH 7.4, was prepared in a 50 mL centrifuge tube. The solution was circulated through the oxygenator following PDA polymerization using a peristaltic pump at 3 mL/min for 24 h. The solution was returned to the tube, creating a closed loop. After ATH polymerization, the oxygenator was washed three times for 5 min each by circulating with PBS, pH 7.4 and followed by using a 3 ml syringe to remove loosely bound molecules.

PDMS-PDA-t-PA oxygenator: 50 mL of a 0.1 mg/mL t-PA solution in PBS, pH 7.4, was prepared in a 50 mL centrifuge tube. The solution was circulated through the oxygenator following PDA polymerization using a peristaltic pump at 3 mL/min for 24 h. The solution was returned to the tube, creating a closed loop. After t-PA polymerization, the oxygenator was washed three times for 5 min each by circulating with PBS, pH 7.4 and followed by using a 3 mL syringe to remove loosely bound molecules.

PDMS-PDA-ATH/t-PA oxygenator: 50 mL of a 0.1 mg/mL ATH and 0.1 mg/mL t-PA mix solution in PBS, pH 7.4, was prepared in a 50 mL centrifuge tube. The solution was circulated through the oxygenator following PDA polymerization using a peristaltic pump at 3 mL/min for 24 h. The solution was returned to the tube, creating a closed loop. After polymerization, the oxygenator was washed three times for 5 min each by circulating with PBS, pH 7.4 and followed by using a 3 mL syringe to remove loosely bound molecules.

### Example 9

### Clotting Time Test of Oxygenator in Plasma

Clot formation and clot lysis in the oxygenator in flowing recalcified plasma were monitored by measuring pressure evolution over time.

Before testing, Citrated plasma and 25 mM calcium chloride were kept at 37° C. in a water bath. 4 mL citrated plasma was mixed with 4 mL 25 mM calcium chloride to prepare recalcified citrated plasma. 7 mL recalcified citrated plasma was injected into the system and circulated for 50 min. The system's pressure was recorded by a pressure monitor at 1 min intervals. ImL recalcified plasma was kept as a control to ensure clot formation.

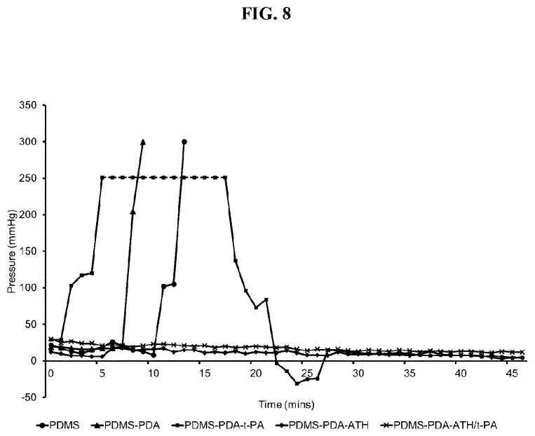

As shown in FIG. 8, the pressure of the unmodified PDMS and PDMS-PDA oxygenators increased at 7 min and 10 min, respectively, indicating clot formation in the oxygenator. The clotting time of the PDMS-PDA sample occurred earlier, which suggests that the PDA coating decreased the coagulation time. The pressure of the t-PA sample increased after 2 min. After 5 min, no-pressure reading was observed on the monitor due to the increased pressure with clot formation. The pressure decreased after about 17 min, suggesting clot lysis. A clot didn't form in the ATH and dual-modified oxygenator, indicating a desired effect of ATH and t-PA on the surface for anticoagulant function.

### Discussion

This application relates to the surface modification of biomaterials with anticoagulant, fibrinolytic and/or antiplatelet molecules to reduce thrombosis. Many single surface modification strategies have been applied to polymer substrates to reduce thrombus formation, including immobilization of the anticoagulant heparin, but these technologies still suffer from various limitations. In the current work, the use of a dopamine-containing bioadhesive on the substrate provides improved results.

In addition, application of a combination of multiple antithrombotic agents is advantageous to achieve a multi-surface modification, such as application of anticoagulant and fibrinolytic strategies, to prevent clot formation as much as possible while lysing any clots that are formed. Nevertheless, functionalizing both on a given surface can alter functionality. In an embodiment described herein, functionalizing a substrate with an anticoagulant agent first, followed by functionalization with a fibrinolytic agent, provides an enhanced response. This response is greater than functionalization with an individual antithrombotic agent over the entire substrate surface and is also surprisingly greater than the response when the fibrinolytic agent is functionalized first.

This invention also provides a method for sequential and simultaneous multi-modification of a substrate with bioactive antithrombotic agents, leading to enhanced surface activity for reducing thrombosis. Applying one bioactive molecule initially provides a base layer to allow the attachment of the second bioactive molecule to cover the remaining surface and/or to attach to the initial bioactive molecule. The overall bioactivity of the biomaterial, thus, can be increased to provide improved antithrombotic properties for medical device applications.

While the present application has been described by reference to specific examples, it is to be understood that the scope of the claims should not be limited by the embodiments outlined in the examples but should be given the broadest interpretation consistent with the description as a whole.

All publications, patents, and patent applications are incorporated by reference in their entirety to the same extent as if each publication, patent or patent application was specifically and individually indicated to be incorporated by reference. Where a term in the present application is defined differently in a document incorporated herein by reference, the definition provided herein is to serve as the definition for the term

### REFERENCES

1. Jaffer I H, Fredenburgh J C, Hirsh J, Weitz J I: Medical device-induced thrombosis: what causes it and how can we prevent it? J Thromb Haemost 2015, 13 Suppl 1: S72-81.
2. Sask K N, Berry L R, Chan A K, Brash J L: Modification of polyurethane surface with an antithrombin-heparin complex for blood contact: influence of molecular weight of polyethylene oxide used as a linker/spacer. Langmuir 2012, 28 (4): 2099-2106.
3. Leung J M, Berry L R, Atkinson H M, Cornelius R M, Sandejas D, Rochow N, Selvaganapathy P R, Fusch C, Chan A K C, Brash J L: Surface modification of poly (dimethylsiloxane) with a covalent antithrombin-heparin complex for the prevention of thrombosis: use of polydopamine as bonding agent. J Mater Chem B 2015, 3 (29): 6032-6036.
4. Leslie D C, Waterhouse A, Berthet J B, Valentin T M, Watters A L, Jain A, Kim P, Hatton B D, Nedder A, Donovan K et al: A bioinspired omniphobic surface coating on medical devices prevents thrombosis and biofouling. Nat Biotechnol 2014, 32 (11): 1134-1140.
5. Biran R, Pond D: Heparin coatings for improving blood compatibility of medical devices. Adv Drug Deliv Rev 2017, 112:12-23.
6. Badv M, Weitz J I, Didar T F: Lubricant-Infused PET Grafts with Built-In Biofunctional Nanoprobes Attenuate Thrombin Generation and Promote Targeted Binding of Cells. Small 2019, 15 (51): e1905562.
7. Zheng Z, Li X, Dai X, Ge J, Chen Y, Du C: Surface functionalization of anticoagulation and anti-nonspecific adsorption with recombinant hirudin modification. Biomaterials Advances 2022.
8. Li D, Chen H, Brash JL: Mimicking the fibrinolytic system on material surfaces. Colloids Surf B Biointerfaces 2011, 86 (1): 1-6.
9. Patel S, Berry L R, Chan A K: Covalent antithrombin-heparin complexes. Thromb Res 2007, 120 (2): 151-160.
10. Martin E. L, Rebecca v d W, fa Almar P, and Brigitte S: Polydopamine—a nature-inspired polymer coating for biomedical science. Nanoscale 2011, 3:4916-4928

1. An antithrombotic surface-modified biomaterial comprising a biomaterial substrate coated with a polymerizable dopamine-containing bioadhesive to which is attached one or more antithrombotic agents.

2. The surface-modified biomaterial of claim 1, wherein the biomaterial is selected from the group consisting of polyurethanes, polydimethylsiloxane (PDMS), polymethylmethacrylate (PMMA), polytetrafluoroethylene (ePTFE), poly (ethylene terephthalate) (PET), polysulfone, polyethersulfone (PES), polypropylene, polyethylene, polyvinylidene fluoride (PVDF), polyvinylchloride (PVC), polyamide and polyetheretherketone (PEEK).

3. The surface-modified biomaterial of claim 1, wherein the biomaterial is a metal substrate.

4. The surface-modified biomaterial of claim 1, wherein the biomaterial is a natural biomaterial substrate selected from the group consisting of biopolyesters, polysaccharides, polypeptides and proteins.

5. The surface-modified biomaterial of any one of claims 1-4, wherein the polymerizable dopamine-containing bioadhesive is polydopamine (PDA) or a mussel foot protein.

6. The surface-modified biomaterial of any one of claims 1-5, wherein the one or more antithrombotic agents are selected from anticoagulant agents, fibrinolytic agents, antiplatelet agents and mixtures thereof.

7. The surface-modified biomaterial of claim 6, wherein the anticoagulant agent is heparin, low molecular weight heparin (LMWH), antithrombin, antithrombin-heparin complex, fondaparinux, rivaroxaban, apixaban, betrixaban, edoxaban, dabigatran, hirudin, bivalirudin, argatroban, thrombomodulin, corn-trypsin inhibitor (CTI) and vitamin K antagonists (VKAs), or a mixture thereof.

8. The surface-modified biomaterial of claim 6 or claim 7, wherein the fibrinolytic agent is tissue plasminogen activator (t-PA), urokinase plasminogen activator (u-PA) or a mixture thereof.

9. The surface-modified biomaterial of any one of claims 6-8, wherein the antiplatelet agent is selected from aspirin, adenosine diphosphate (ADP) receptor inhibitors, GPIIb/IIIa inhibitors, dipyridamole, prostacyclin, apyrase and mixtures thereof.

10. The surface-modified biomaterial of any one of claims 1-8, wherein the one or more antithrombotic agents comprise an anticoagulant agent and a fibrinolytic agent.

11. The surface-modified biomaterial of claim 10, wherein the anticoagulant agent comprises heparin and the fibrinolytic agent comprises t-PA.

12. The surface-modified biomaterial of claim 10, wherein the anticoagulant agent comprises antithrombin-heparin complex and the fibrinolytic agent comprises t-PA.

13. The surface-modified biomaterial of any one of claims 1-12, wherein the one or more antithrombotic agents are each attached directly to the bioadhesive coated substrate.

14. The surface-modified biomaterial of any one of claims 1-12, comprising a monolayer of one antithrombotic agent on the bioadhesive coated substrate and at least a second antithrombotic agent attached to the surface of the monolayer.

15. A method of preparing an antithrombotic surface-modified biomaterial as defined in any one of claims 1-14, said method comprising the steps of:
i) contacting a biomaterial substrate with a dopamine-containing bioadhesive under conditions sufficient to polymerize the bioadhesive as a coating on the biomaterial substrate, and
ii) contacting the polymerized dopamine-containing coating with a quantity of one or more antithrombotic agents under conditions sufficient to achieve attachment of the antithrombotic agent.

16. The method of claim 15, wherein two or more antithrombotic agents are contacted with the polymerized dopamine-containing coating simultaneously.

17. The method of claim 15, wherein two or more antithrombotic agents are contacted with the polymerized dopamine-containing coating sequentially.

18. The method of claim 16 or claim 17, wherein the antithrombotic agents comprise an anticoagulant agent and a fibrinolytic agent.

19. The method of claim 17, wherein the antithrombotic agents comprise an anticoagulant agent and a fibrinolytic and/or an antiplatelet agent, and the anticoagulant agent is contacted with the dopamine-containing coating first, followed by sequential contact of the fibrinolytic and/or antiplatelet agents with the dopamine-containing coating.

20. The method of claim 16, wherein the antithrombotic agents comprise the anticoagulant agent and an antiplatelet agent.

21. The method of claim 19, wherein the antithrombotic agents comprise the anticoagulant agent and a fibrinolytic agent.

22. The method of claim 16 or claim 17, wherein the antithrombotic agents comprise an anticoagulant agent, a fibrinolytic agent and an antiplatelet agent.

23. The method of any one of claims 15-22, wherein the anticoagulant agent is unfractionated heparin, low molecular weight heparin or an antithrombin-heparin covalent complex.

24. The method of any one of claims 15-23, wherein the fibrinolytic molecule is tissue plasminogen activator (tPA).

25. The method of any one of claims 15-24, wherein the dopamine-containing bioadhesive is polydopamine (PDA).

26. A blood contacting devices comprising an antithrombotic surface-modified biomaterial as defined in any one of claims 1-14.

27. The device of claim 26, which is selected from the group consisting of a catheter, guidewire, dialyzer, oxygenator, heart-supporting system, cardiac pacemaker, vascular graft, stent, heart valve, blood pump, suture, microparticle, nanoparticle, scaffold for containing cells or tissue, orthopedic implant and dental implant.
